(19) United States
(12) Patent Application Publication
(10) Pub. No.: US 2004/0092577 A1
(43) Pub. Date: May 13, 2004

(54) Microparticle pharmaceutical compositions for intratumoral delivery

(76) Inventors: E. Itzhak Lerner, Petach Tiqva (IL); Moshe Flashner-Barak, Petach Tikva (IL); Abraham R. Tzafriri, Jerusalem (IL); Hanna Parness, Jerusalem (IL); Alan Smith, Nottingham (GB); Michael Hinchcliffe, Nottingham (GB)

Correspondence Address: KENYON & KENYON, ONE BROADWAY, NEW YORK, NY, 10004, US

(21) Appl. No.: 10/423,345

(22) Filed: Apr. 24, 2003

Related U.S. Application Data
(60) Provisional application No. 60/376,080, filed on Apr. 26, 2002.

(51) Int. Cl. A61K 31/337; A61K 9/50

(52) U.S. Cl. 514/449; 424/499

(57) ABSTRACT

Provided are microparticles including paclitaxel, methods for making them, and pharmaceutical compositions containing them. Also provided are methods of treating tumors including the step of intratumorally injecting the paclitaxel-containing microspheres of the present invention.

### RELATED APPLICATIONS

The present application claims the benefit of the filing date of U.S. Provisional Patent Application Serial No. 60/376,080 filed Apr. 26, 2002.

### FIELD OF THE INVENTION

The present invention relates to novel pharmaceutical compositions of antineoplastic drugs, especially paclitaxel, and to novel methods of treating solid tumors using these pharmaceutical compositions.

### BACKGROUND OF THE INVENTION

Surgical excision is a very common course of treatment for a mammal, especially a human, having a solid tumor, especially a malignant solid tumor. Examples of solid tumors include myeloid sarcomata, round-celled sarcromata, melanotic sarcoma, spindle-cell sarcoma, and papillomata, to mention just a few. Other types of solid tumors are well known to one skilled in the medical arts.

Frequently, the practitioner is confronted with a situation in which a solid tumor cannot be excised, that is, the solid tumor is inoperable. A solid tumor can be inoperable because of its location, or it can be inoperable because of its size. Chemotherapy is often used in the treatment of solid tumors to shrink their size, thereby rendering them operable.

At least three general methods of chemotherapy are known: (1) systemic intravenous (IV), (2) intra-arterial, and (3), intratumoral. Each of these has advantages and disadvantages.

Systemic preoperative I.V. therapy has been found to be effective in reducing or shrinking a solid tumor (Ferriere, J. P. et. al. Primary chemotherapy in breast cancer: Correlation between tumor response and patient outcome, Am. J. Clin. Oncol. Cancer Clin. Trials 1998, 21(2), 117-120 ). Moreover, the I.V. route gives concurrent treatment to the entire organism so that metastatic cells (or micrometastases) are being treated throughout the body. However, obstacles exist which reduce the effectiveness of this treatment method. The major obstacle is attainment of an effective concentration for therapy, that is, getting enough antineoplastic agent to the tumor. Due to the cytotoxic nature of the drugs that are being distributed throughout the organism, systemic chemotherapy can cause side effects, Sometimes, chemotherapy becomes intolerable for the patient, limiting the use of a particularly powerful drug. According to the literature, most drugs are administered systemically at the limit of tolerable side effects (MTD-maximum tolerated dose), at doses which do not provide optimum efficacy.

This limitation to the MTD not only affects success of the treatment, but also may have the counterproductive result of forming a more resistant tumor. It is assumed that there are several populations of the same type of tumor cell within a specific solid tumor that differ from one another by their ability to resist a chemotherapeutic agent at a particular dose level. Kinsella, A. R. et. al. Resistance to chemotherapeutic antimetabolites: A function of salvage pathway involvement and cellular response to DNA damage, Br. J. Cancer 1997, 75(7), 935-945. The MTD may be a dose level which is capable of killing most, but not all, of the cells in the particular tumor. As a result, not only do residual amounts of cancer cells remain but also, due to extensive proliferation; those new cells are reported to dominate most of the tumor and will provide a more difficult challenge for treating that tumor chemically in the future. Another obstacle is the fact that many antineoplastic drugs may be phase sensitive. That is, they interact with the cells only when the cells are in a particular stage of the cell cycle. Other cells, not in the sensitive stage at the time of dosing, are spared. I.V. dosing, being of relatively short duration, may miss the sensitive phase of the tumor cells even when giving high dose intensity. Many tumors could benefit from a lower dose, high frequency or continuous dosing schedule both in efficacy and in lowering adverse event intensity.

Paclitaxel, also known as Taxol®, is an example of a reportedly phase sensitive antineoplastic drug that could be used more efficaciously by frequent lower dosing or extended dosing as opposed to intermittent higher dosing.

Intra-arterial chemotherapy was introduced as an attempt to address the problem of dosing at the MTD and not necessarily at the most effective dose. The concept behind this approach is that by administering the drug into the arterial blood flow in the target area, very high local concentrations of the drug will be produced in the solid tumor. The dose will be diluted by the blood flow after leaving the area of the solid tumor, thereby avoiding or mitigating side effects. This method has been successfully tested in what are known to be resistant tumors. Tang, Z. Y. , Hepatocellular carcinoma, J. Gastroenterol. Hepatol. 2000, 15, G1-G7; Takashima, S. et. al. Means of effective and practical intra-arterial chemotherapy for locally invasive bladder cancer—With special reference to clinical analysis of bladder cancer patients treated by intermittent intra-arterial infusion using an implantable port system, Acta Urol. Jpn., 1999, 45(2) 127-131. In other cases, the clearance of the drug and its subsequent dilution was too effective to allow enhanced treatment by this method.

A reported major problem with intra-arterial chemotherapy is its complexity, requiring a high level of skill in the treating practitioner, and the need for sophisticated equipment. Serious side effects have resulted if the procedure is not performed correctly. Tonus, C. et. al., Complications of intra-arterial chemotherapy for liver metastases from colorectal carcinoma, Curr. Oncol., 2000, 7(2), 115-118; Arai, K. et al. , Complications related to catheter indwelling in intra-arterial infusion chemotherapy from the standpoint of the route of canulation, Jpn. J. Cancer Chemother., 1992, 19(10), 1568-1571. As a result, intra-arterial therapy has been limited in its application. The method also does not address the issue of the phase sensitive nature of many cytotoxic drugs. On the other hand, it has helped to overcome the problem of tumor resistance. The studies performed with intra-arterial delivery have demonstrated that a high enough concentration of a chemotherapeutic agent would eliminate the tumor totally, regardless of the “resistance” to a previous systemic chemotherapy.

If and when a more friendly intra-arterial procedure is developed, it will provide the practitioner with a method of administering the drug close to the tumor with fewer complications. At that stage, this technique of chemotherapy may replace systemic IV chemotherapy.

Intratumoral injection is a promising alternative technique for chemotherapy and, at least conceptually, should present the most successful approach. In this method, the antineoplastic drug is administered directly to the tumor, thus achieving high local concentrations and avoiding systemic side effects. This method also provides an almost infinite flexibility in dosage.

In spite of all these advantages, intratumoral chemotherapy has not been particularly effective. It has been proposed that the reasons for this lack of efficacy are due to one or more of the following factors:

The density of the tumor cells in the tumor is very high, thus preventing drug penetration through the cells when it is not via the blood vessels,

The interstitial fluid pressure is high, preventing migration of the drug into the interstitial fluid,

The high density of cells and blood vessels causes the blood vessels themselves to constrict.

See (Jain, R. K., Transport of molecules, particles and cells in solid tumors, Annu. Rev. Biomed. Eng., 1999, 01, 241-263). Proposals as to dosing protocols to alleviate these problems by inducing apoptosis in the tumor have been advanced. See, e.g., M. Flashner-Barak, U.S. patent application Ser. No. 2002/0041888 A1, Ser. No. 09/829,621.

Other possible reasons for failure of intratumoral dosing have been proposed; including non-homogeneous spread of the drug throughout the tumor and the lack of an effective dose for a long enough period to treat the cells when they enter their sensitive phase in the cycle. The problem in intratumoral chemotherapy then reduces to maintaining a high enough concentration of a chemotherapeutic agent over a long enough time period, spread throughout the tumor, in order to achieve these goals.

Intratumoral injections have been carried out using gels, pastes and microparticles. Paclitaxel has been incorporated into gels at 0.6% loading and used intratumorally. The release rates were such to give delivery from 1 to 6 weeks. Zentner, G. M. et. al., Biodegradable block copolymers for delivery of proteins and water-insoluble drugs, J. Control. Release, 2001, 72(1-3), 203-215. Paclitaxel has been incorporated into pastes of poly(lactic acid) (PLA ) and poly (ε-caprolactone) and injected intratumorally. The release rate was about 100 μg/day. Jackson, J. K. et. al., The suppression of human prostate tumor growth in mice by the intratumoral injection of a slow-release polymeric paste formulation of paclitaxel, Cancer Res. 2000, 60(15), 4146-4151. Paclitaxel has been incorporated into microspheres at 10-30% loading in PLA with ˜25% of the drug being released over 30 days. Ligins, R. T., et. al., Paclitaxel loaded poly(L-lactic acid) microspheres for the prevention of intraperitoneal carcinomatosis after a surgical repair and tumor cell spill, Biomaterials, 2000, 21(19), 1959-1969. Paclitaxel has also been incorporated at 10% loading in microspheres of PACLIMER® polymer with drug release of 80% over 90 days for intratumoral injection into lung cancer nodules, Harper, E. et al., Enhanced efficacy of a novel controlled release paclitaxel formulation (PACLIMER delivery system) for local-regional therapy of lung cancer tumor nodules in mice, Clin. Canc. Res. 1999, 5(12), 4242-4248; at 5% loading in poly(ε-caprolactone) releasing 25% of the drug in 6 weeks (Dordunoo, S. K., et al., Taxol encapsulation in poly(ε-caprolactone) microspheres, Cancer Chemother. Pharmacol. 1995, 36(4), 279-282); and at 0.6% loading in a blend of ethylene-vinyl acetate copolymer with PLA with ˜10% of the drug being released in 50 days, Burt, H. M., et. al. , Controlled delivery of Taxol from microspheres composed of a blend of ethylene-vinyl acetate copolymer and poly(d,l lactic acid), Cancer Lett. 1995, 88(1), 73-79. Paclitaxel has also been incorporated in microspheres at 2% loading in poly (lactic-co-glycolic acid) (PLGA), giving release of up to 50% of the drug in 100 days depending on the formulation. Mu, L. and Feng, S. S., Fabrication, characterization and in vitro release of paclitaxel (Taxol) loaded poly(lactic-co-glycolic acid) microspheres prepared by spray drying technique with lipid/cholesterol emulsifiers, J. Control. Release, 2001, 76(3), 239-254.

Similarly, paclitaxel has been incorporated at 30% loading in PLA of various molecular weights giving molecular weight dependent release of between 11 to 76% in 14 days. Liggins, R. T. and Burt, H. ., Paclitaxel loaded poly(L-lactic acid) microspheres: Properties of microspheres made with low molecular weight polymers, Int. J. Pharm. 2001, 222(1), 19-33. Paclitaxel has also been incorporated in nanospheres of PLGA, Feng, S. S. et. al., Nanospheres of biodegradable polymers,: A system for clinical administration of an anticancer drug paclitaxel (Taxol), Ann. Acad. Med, Singapore, 2000, 29(5), 633-639, and in nanospheres of polyvinylpyrrolidone (PVP) for I.V. administration where the concentration of the paclitaxel was 0.3% in the suspension, Sharma, D. et. al. Novel Taxol® formulation: Polyvinylpyrrolidone nanoparticle-encapsulated Taxol® for drug delivery in cancer therapy, Oncology Research, 1996, 8(7/8), 281-286. Paclitaxel has been incorporated at 2.8% loading in solid lipid nanospheres exhibiting a very slow release profile. Cavalli, R., et. al., Preparation and characterization of solid lipid nanospheres containing paclitaxel, Eur. J. Pharm. Sci., 2000, 1(4), 305-309.

In all the foregoing studies, the paclitaxel reportedly showed some efficacy, but responses were only moderate. One may speculate that the gels and pastes do not spread homogeneously throughout the tumors. Use of microspheres might alleviate that problem. In the above-cited studies, the microspheres were all designed and formulated to give extended release over long periods of time and, therefore, should have been able to cover all phases of the cell cycle efficiently. However, the reported results were not as good as hoped for.

As discussed above, the prior art teaches that, for intratumoral injection, the antineoplastic agent should be released over a relatively long period of time. The present inventors have discovered that this widely-shared conventional wisdom is wrong and that long-term release of antineoplastic drug at the site of intratumoral injection is counterproductive. The present inventors have discovered that an optimum intratumoral release profile for poorly water soluble antineoplastic drugs like paclitaxel, resulting in maximum cell kill, can be achieved by using microparticles of a particular size and made with a water soluble polymer. The present inventors have also developed a theoretical model (the model) that, while not limiting the invention in any way, rationalizes this unexpected result.

### SUMMARY OF THE INVENTION

In one aspect, the present invention relates to a pharmaceutical powder that can be constituted to a pharmaceutical composition for intratumoral injection wherein the powder includes microparticles that have from about 50% to about 90% by weight of an antineoplastic drug that is poorly soluble in water, especially paclitaxel, the remainder of the microparticle having at least one water soluble polymer.

In another aspect, the present invention relates to a pharmaceutical powder that can be constituted to a pharmaceutical composition for intratumoral injection wherein the powder includes microparticles that have from about 50% to about 90% by weight of an antineoplastic drug that is poorly soluble in water, especially paclitaxel, the remainder of the microparticle having at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides.

In another aspect, the present invention relates to a pharmaceutical powder that can be constituted to a pharmaceutical composition for intratumoral injection wherein the powder includes microparticles that have from about 50% to about 90% by weight of an antineoplastic drug that is poorly soluble in water, especially paclitaxel, the remainder of the microparticle having at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides, wherein the microparticles have an average nominal diameter between about 0.5μ and about 10μ.

In another aspect, the present invention relates to a pharmaceutical powder that can be constituted to a pharmaceutical composition for intratumoral injection wherein the powder includes microparticles that have from about 50% to about 90% by weight of an antineoplastic drug that is poorly soluble in water, especially paclitaxel, the remainder of the microparticle having at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides and further including at least one emulsifier or surface active agent.

In another aspect, the present invention relates to a pharmaceutical powder that can be constituted to a pharmaceutical composition for intratumoral injection wherein the powder includes microparticles that have from about 65% to about 75% by weight of an antineoplastic drug that is poorly soluble in water, especially paclitaxel, the remainder of the microparticle having at least one water soluble polymer, wherein the particles have an average diameter between about 1μ and about 10μ.

In yet another aspect, the present invention relates to a pharmaceutical powder, capable of being constituted to a pharmaceutical composition for intratumoral injection, comprising microparticles having an average diameter between about 2μ and about 4μ wherein the microparticles comprise from between about 65% by weight to about 75% by weight, based on the weight of microparticles, of paclitaxel and between about 25% by weight and about 35% by weight, based on the weight of microparticles, of polyvinylpyrrolidone.

In a further aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer.

In another aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer and at least one emulsifier and/or surface active agent.

In another aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer, wherein the microparticles have an average nominal diameter between about 0.5μ and about 10μ.

In another aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides, wherein the microparticles have an average nominal diameter between about 0.5μ and about 10μ.

In another aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides and further including at least one emulsifier or surface active agent, wherein the microparticles have an average nominal diameter between about 0.5μ and about 10μ.

In still another aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer, wherein the microparticles have an average nominal diameter between about 1μ and about 5μ.

In still another aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer, wherein the microparticles have an average nominal diameter between about 1μ and about 5μ and wherein the particles are present in the pharmaceutical composition in a concentration of between about 20 mg/ml and about 300 mg/ml.

In still another aspect, the present invention relates to a pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer, wherein the microparticles have an average nominal diameter between about 1μ and about 5μ and wherein the particles are present in the pharmaceutical composition in a concentration of between about 200 mg/ml and about 300 mg/ml.

In yet another aspect, the present invention relates to a pharmaceutical composition, suitable for intratumoral injection, comprising microparticles having an average diameter between about 2μ and about 4μ wherein the microparticles comprise from between about 65% by weight to about 75% by weight, based on the weight of microparticles, of paclitaxel and between about 25% by weight and about 35% by weight, based on the weight of microparticles, of polyvinylpyrrolidone.

In yet a further aspect, the present invention relates to a pharmaceutical composition, suitable for intratumoral injection, comprising microparticles wherein the microparticles comprise from about 50% by weight to about 75% by weight, based on the weight of microparticles, of paclitaxel, the remainder by weight of the microparticles comprising at least one water soluble polymer, wherein upon intratumoral injection of the composition the microparticles spread in the tumor wherefrom paclitaxel is released in a therapeutically effective amount in an extended manner for between about 24 and about 240 hours.

In another aspect, the present invention provides a method of treating a solid tumor comprising the step of intratumorally injecting a pharmaceutical composition wherein the pharmaceutical composition comprises microparticles, wherein the microparticles comprise from about 50% by weight to about 90% by weight of paclitaxel, the remainder by weight of the microparticles comprising at least one water soluble polymer.

In another aspect, the present invention relates to a method of treating a solid tumor comprising the step of intratumorally injecting a pharmaceutical composition wherein the pharmaceutical composition comprises microparticles, wherein the microparticles comprise from about 50% by weight to about 90% by weight of paclitaxel, the remainder by weight of the microparticles comprising at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides.

In another aspect, the present invention relates to a method of treating a solid tumor comprising the step of intratumorally injecting a pharmaceutical composition wherein the pharmaceutical composition comprises microparticles, wherein the microparticles comprise from about 50% by weight to about 90% by weight of paclitaxel, the remainder by weight of the microparticles comprising at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides and wherein the microparticles have and average diameter between about 2μ and about 4μ.

In [JBS1]still another aspect, the present invention relates to a method of treating a solid tumor comprising the step of intratumorally injecting a pharmaceutical composition wherein the pharmaceutical composition comprises microparticles, wherein the microparticles comprise from about 50% by weight to about 90% by weight of paclitaxel, the remainder by weight of the microparticles comprising at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides, wherein the paclitaxel is released in an extended manner for between about 24 and about 240 hours.

In [JBS2]still yet a further aspect, the present invention relates to a method of treating a solid tumor selected from the group consisting of breast tumor, ovarian tumor, head and neck tumors, tumors of the peritoneal cavity, testicular tumors, tumors of the rectum, and pancreatic tumors; comprising the step of intratumorally injecting a pharmaceutical composition wherein the pharmaceutical composition comprises microparticles, wherein the microparticles comprise from about 50% by weight to about 90% by weight of paclitaxel, the remainder by weight of the microparticles comprising at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides, wherein the paclitaxel is released in an extended manner for between about 24 and about 240 hours.

In still yet another aspect, the present invention relates to a method of treating a solid tumor comprising the step of intratumorally injecting a pharmaceutical composition wherein the pharmaceutical composition comprises microparticles, wherein the microparticles comprise from about 50% by weight to about 90% by weight of paclitaxel, the remainder by weight of the microparticles comprising at least one water soluble polymer selected from the group consisting of polyvinylpyrrolidone, hydroxypropylcellulose, methylcellulose, hydroxypropylmethylcellulose, sodium carboxymethylcellulose, hydroxyethylcellulose, and polysaccharides, wherein the paclitaxel is released in an extended manner for between about 48 and about 100 hours.

### BRIEF DESCRIPTION OF THE DRAWINGS

FIG. 1 illustrates the extracellular concentration vs. time curve for different values of Tₘₐₓ.

FIG. 2 shows extracellular drug concentration as a function of time for different microparticle loadings.

FIG. 3 shows the effect of an initial extracellular drug concentration.

FIG. 4 shows tumor area vs time for various tumor treatments.

### THEORETICAL CONSIDERATIONS

The present inventors have discovered that the release profile (i.e. extracellular concentration vs. time) achieved in the inventive methods using their inventive pharmaceutical compositions can be rationalized by a reaction diffusion model described below.

The principal processes governing drug transport inside a solid tumor are: (1) diffusion and binding in the extracellular medium, (2) drug clearance from the extracellular medium through the leaky microvessels, (3) passive uptake of free extracellular drug by the intracellular medium and (4) specific and non-specific binding of drug in the intracellular medium. The present model can be extended to consider drug metabolism in either of the mediums, intracellular drug diffusion, and active efflux from the cells, if necessary.

The model incorporates several approximations. First, the model focuses on a representative spherical section of the tumor of radius R_{K}, which contains at least one microsphere. Second, convection is neglected (only drug clearance need be modeled). Third, the flux of drug released from the microspheres is a known function of time. The first approximation is similar to the notion of Krough cylinders in models of transvascular delivery. The radius of such a Krough sphere, R_{K}, must be much smaller than the tumor radius, R_{T}, in order to justify the notion of a representative section of the tumor bulk. Conversely, in order to justify a continuum approach, R_{K} should be large enough so that it contains many cells and microspheres. These two opposing restrictions on R_{K} can be met when the microsphere density is sufficiently high. Ignoring explicit convection effects is justified whenever the timescale for convection is orders of magnitude longer than the timescale for discussion [1]. We believe this is the case when paclitaxel is the antineoplastic drug because paclitaxel is a small fast diffusing molecule, intratumoral fluid flow is slow, and R_{K}<<R_{T}. Finally, the assumption of a uniform source of drug from the microspheres was shown to be attainable under realistic conditions.

The following assumptions and boundary conditions are used in the model:

1. the geometry is stationary since tumor growth is very slow,

2. the tumor is macroscopically homogeneous with respect to cell and micro-vessel distribution,

3. the intracellular gaps are sufficiently large to allow a uniform distribution of injected microspheres,

4. the tumor is sufficiently large compared to the microspheres and cells so that surface effects can be neglected,

5. only a homogeneous spherical portion of the tumor is considered and interaction between microspheres is neglected [2]. This is similar to the notion of Krough cylinders [3, 4] in models of trans-vascular drug delivery to tumors and we also use symmetry boundary conditions at the surface of the sphere,

6. as a first approximation, the effects of cell cycle effects (e.g., tubulin kinetics) and cell kill kinetics (e.g., apoptosis) on the transport of the drug in the extracellular matrix and the uptake of drug by the intracellular matrix are neglected,

7. drug can bind reversibly to proteins (i.e. there is one type of saturable binding sites in the extracellular medium and two types of intracellular binding sites: saturable and non-saturable) [5],

8. drug absorption by the cells is passive, e.g., there are no active pumps at the cell surface. This assumption is easily relaxed as long as the competing absorption and efflux mechanisms are additive (for example [6]), and

9. all the relevant processes can be described using reaction diffusion equations with appropriate initial conditions and boundary conditions (and possibly source or sink terms).

The important assumption here is that the detailed modeling of convection effects can be neglected. This is justified by the high extracellular diffusion coefficient of paclitaxel [7] and the relatively small diffusive path considered here [1]. This assumption has to be reconsidered critically when modeling the whole tumor.

With the above approximations and assumptions, the following equations can be written.

$$\begin{matrix}\frac{ⅆ W}{ⅆ t} = - {μ}_{0} , r < {R}_{m} , & ( 1 ) \\ - {D}_{e} ∇ {C}_{e} = \frac{1}{4 π {R}_{m}^{2}} \frac{ⅆ W}{ⅆ t} , r = {R}_{m} , & ( 2 ) \\ \frac{\partial {C}_{e}}{\partial t} - {D}_{e} Δ {C}_{e} = - \frac{\partial {B}_{e}}{\partial t} - α ( {C}_{e} - {C}_{i} ) - γ {C}_{e} , {R}_{m} < r < {R}_{K} , & ( 3 ) \\ \frac{\partial {B}_{e}}{\partial t} = {k}_{e , a} ( {B}_{e , max} - {B}_{e} ) - {k}_{e , d} {B}_{c} , {R}_{m} < r < {R}_{K} , & ( 4 ) \\ \frac{\partial {C}_{i}}{\partial t} = - \frac{\partial {B}_{i1}}{\partial t} - \frac{\partial {B}_{i2}}{\partial t} + α ( {C}_{e} - {C}_{i} ) , {R}_{m} < r < {R}_{K} , & ( 5 ) \\ \frac{\partial {B}_{i1}}{\partial t} = {k}_{i1 , a} {C}_{i} ( {B}_{i1 , max} - {B}_{i1} ) - {k}_{i1 , d} {B}_{i1} , {R}_{m} < r < {R}_{K} , & ( 6 ) \\ \frac{\partial {B}_{i2}}{\partial t} = {k}_{i2 , a} {C}_{i} ( {B}_{i2 , max} - {B}_{i2} ) - {k}_{i2 , d} {B}_{i2} , {R}_{m} < r < {R}_{K} , & ( 7 )\end{matrix}$$

If boundary conditions at the surface of the sphere are symmetrical then;

∇Cₑ=0, r=R_{K} (8)

and assuming uniform initial conditions;

Cₑ=C₀, t=(1 and Rₘ<r<R_{K}, (9)

Bₑ=Cᵢ=Bᵢ₁=Bᵢ₂=0, t=0 and Rₘ<r<R_{K}. (10)

In the foregoing equations, the following variables have the indicated meaning. Rₘ and R_{K} are, respectively, the microsphere and “Krough” sphere radii, Cₑ and Bₑ are, respectively the free and bound extracellular drug concentrations; Cᵢ is the intracellular concentration of free drug and, Bᵢ₁ and Bᵢ₂ are, respectively, the concentrations of specifically and non-specifically intra-cellularly bound drug, α is the (passive) cell permeability of the drug; γ is the rate of drug clearance from the extracellular medium (due to microvessels); D_{c} is the drug diffusion coefficient in the extracellular medium; B_{e,max} k_{e,a} and k_{e,d} are the drug binding parameters in the extracellular medium; B_{i1,max} k_{i1,a} and k_{i1,d}, are the parameters of drug binding to the saturable sites in the intracellular medium; B_{i2,max} k_{i2,a} and k_{i2,d} are the parameters of drug binding to the non-saturable sites in the intracellular medium.

We divide the parameters into two groups: Table 1 lists the range of model parameters which are of conceptual importance, whereas Table 2 lists the range of parameter values which are actually used in the simulation of the model, Eqs. (1)-(10). The estimate of R_{K} is based on the identity.

R_{K}=R_{T}N^{-1/3}. (11)

The zero order drug release rate, μ₀, can be estimated from the following relation,

$${μ}_{0} = \frac{{W}_{load}}{{A}_{d} {V}_{m} {T}_{max}} , \qquad ( 12 )$$

where W_{load} is the drug load, Vₘ is the microsphere volume, A_{d} is the molecular weight of paclitaxel and Tₘₐₓ is the duration of drug release from the microsphere. W_{load} was estimated by assuming a drug load of 5-30% w/w. The estimate of Tₘₐₓ is based on poly(lactic-co-glycolic acid) microspheres containing isopropyl myristate [8]. The default value of μ₀ appearing in Table 2 corresponds to a 20% drug load (W_{load}=1.3 pg) and Tₘₐₓ=100 h.

The maximal tissue diffusion coefficient of paclitaxel is taken from the literature [7]. The minimal value is due to hindrance by the extracellular matrix [9]. According to El-Karch et al. [10], hindrance effects are unimportant for small molecules such as paclitaxel, and the volumetric hindrance depends on the volume fraction approximately as:

$$D / {D}_{0} ≃ \frac{2 φ}{3 - φ} . \qquad ( 13 )$$

Estimates of the interstitial volume fraction, φ, are from Jain [11].

TABLE 1
Range of important model parameter values.

| parameter | meaning | maximum | minimum |
|---|---|---|---|
| R_{T}(cm) | tumor radius | 1.0 | 0.25 |
| N | No. of microspheres | 10¹⁰ | 10⁶ |
| Tₘₐₓ(h) | duration of release | 240 | 48 |
| Vₘ(pL) | microsphere volume | 0.004 | 0.004 |
| ρₘ(g/mL) | microsphere density | 1.5 | 1.0 |
| Wₘ(pg) | microsphere weight | 6.0 | 4.0 |
| W_{load}(pg) | drug load | 2.0 | 0.2 |
| A_{d} | MW of drug | 820 | 820 |
| φ | interstitial v.f. | 0.55 | 0.13 |
| Cₛ(μM) | aq. solubility | 35 | 0.5 |
| Cₜₕ(μM) | therapeutic conc. | 6.0 | 0.1 |

TABLE 2
Range of values of parameters used in simulating Eqs. (1)-(10).
See text for explanations.

| parameter | maximum | minimum | default |
|---|---|---|---|
| R_{K}(μm) | 45 | 5 | 10 |
| Rₘ(μm) | 1 | 1 | 1 |
| μ₀(μMh⁻¹) | 12,700 | 254 | 4,000 |
| Dₑ(cm²/s) | 1 × 10⁻⁶ | 1 × 10⁻⁷ | 1 × 10⁻⁷ |
| α(h⁻¹) | 2160 | 58 | 1,800 |
| γ(h⁻¹) | 180 | 36 | 36 |
| B_{e.max}(μM) | 5 | 3 | 5 |
| Kₑ(μM⁻¹) | 1.35 | 1.25 | 1.35 |
| k_{e,d}(h⁻¹) | 14.4 | 1.4 | 14.4 |
| B_{i1,max}(μM) | 70 | 60 | 70 |
| Kᵢ₁(μM⁻¹) | 250 | 100 | 250 |
| k_{i1,d}(h⁻¹) | 14.4 | 1.4 | 14.4 |
| B_{i2,max}Kᵢ₂ | 0.18 | 0.12 | 0.18 |
| k_{i2,d}(h⁻¹) | 10,800 | 540 | 10,800 |

The rate of passive uptake, α, is estimated from the literature. Kuh et al. [5] estimated α=0.47±0.1 3s⁻¹ for Taxol® uptake by human breast adenocarcinoma MCF7 cells which have negligible pGp expression. Lankmela et al. estimated α=0.016s⁻¹ for doxorubicin uptake by MDA-468 breast cancer cells [12]. The discrepancy is probably due to the high lipophilicity of paclitaxel [13, 14]. Drug clearance rate from the extracellular medium, γ, is estimated from published values of venous appearance rate following intratumoral drug infusion [15]. Note, that α∝σ_{c} and γ∝σₘᵥ, where σ_{c} and σₘᵥ are the specific surface areas of the cells and microvessels, respectively. According to the literature, σ_{c}≈4700 cm⁻¹ [12] and σₘᵥ≈200 cm⁻¹ [16]. We would therefore expect

$$\frac{α}{γ} \approx \frac{{σ}_{c}}{{σ}_{mv}} \approx 23.$$

Estimates of the equilibrium parameters of non-specific binding, B_{e,max}, Kₑ=k_{e,a}/k_{e,d}, B_{i1,max} and Kᵢ₁=k_{i1,a}/k_{i1,d}, are taken from [5]. K_{i2,d}=k_{i2,a}/k_{i2,d} is taken from the literature [17]. In the absence of kinetic data for drug binding to the extracellular medium we estimated k_{e,d} by analyzing the non-specific binding of Taxol® onto glass containers [18]. The parameters for specific (linear) intracellular binding medium are estimated from published data (Kᵢ₂Bᵢ₂ₘₐₓ from [5] and k_{i2,d} from [16].

Drug solubility [19] is not used in the model, but it is important to verify that the predicted free drug concentrations do not approach the solubility limit. Similarly, the therapeutic concentration, Cₜₕ, is important for analyzing the relevance of our results according to the clinical case. Here, Cₜₕ is defined as the range of extracellular paclitaxel concentrations which has significant pharmacodynamic efficacy. Estimates of Cₜₕ are taken from the literature [20].

Significant events occur on different time scales. The time scale for diffusion of drug in extracellular medium can be expressed as:

$${T}_{D} ≡ \frac{{R}_{K}^{2}}{{D}_{e}} . \qquad ( 14 )$$

Using the default values shown in Table 2 we estimate the time scale for drug diffusion

$${T}_{D} ≃ \frac{{10}^{- 6} {cm}^{2}}{{10}^{- 7} {cm}^{2} \text{/} s} = 10 s = 0.03 h .$$

The initial time scales for binding can be expressed as:

$$\begin{matrix}{T}_{B , e} = \frac{1}{{k}_{e , a} {B}_{e , max}} = \frac{1}{{k}_{e , d} {K}_{e} {B}_{e , max}} , & ( 15 ) \\ \begin{matrix}{T}_{B , i1} = \frac{1}{{k}_{i1 , a} {B}_{i1 , max}} = \frac{1}{{k}_{i1 , d} {K}_{i1} {B}_{i1 , max}} \\ and\end{matrix} & ( 16 ) \\ {T}_{B , i2} = \frac{1}{{k}_{i2 , a} {B}_{i2 , max}} = \frac{1}{{k}_{i2 , d} {K}_{i2} {B}_{i2 , max}} . & ( 17 )\end{matrix}$$

Using the default values shown in Table 2, we estimate the time scale for the various types of binding as:

$${T}_{B , e} ≃ \frac{1}{14.4 {h}^{- 1} \times 1.35 \times 5} = 0.01 h . \\ {T}_{B , i1} ≃ \frac{1}{14.4 {h}^{- 1} \times 250 \times 70} = 4 \times {10}^{- 6} h , \\ {T}_{B , i2} ≃ \frac{1}{10 , 800 {h}^{- 1} \times 0.18} = 5 \times {10}^{- 4} h .$$

Based on the foregoing, we conclude that drug release from the microparticle is the rate-limiting step. Accordingly, the dynamics of intratumoral drug concentration can be divided into an initial transient, during which diffusion is important, followed by a spatially homogeneous quasi steady-state.

During the quasi steady-state asymptotic diffusion, binding and cellular uptake are negligible so that Eqs. (1)-(10) can be simplified to:

$$\begin{matrix}\frac{ⅆ W}{ⅆ t} = - {μ}_{0} , r < {R}_{m} , & ( 20 ) \\ {V}_{K} \frac{d {C}_{e}}{\partial t} = - γ {V}_{K} {C}_{e} + {V}_{m} {μ}_{0} = , {R}_{m} < r < {R}_{K} . & ( 21 )\end{matrix}$$

Since the long time asymptotic begins after the saturation of binding sites, Equation (21) has to be solved subject to the following initial conditions:

Cₑ=0, t=Tᵣᵢₛₑ and Rₘ<r<R_{K}. (22)

Thus,

$${C}_{e} = \frac{μ}{γ} ( 1 - {ⅇ}^{- γ ( t - {T}_{rise} )} ) , t > {T}_{rise} , \qquad ( 23 )$$

where we introduced the simplifying notation

$$μ ≡ \frac{{V}_{m} {μ}_{0}}{{V}_{k}} = \frac{{W}_{load}}{{A}_{d} {V}_{K} {T}_{max}} . \qquad ( 24 )$$

Moreover, the following estimate is obtained for the initial transient which precedes the saturation of binding sites

$${T}_{res} ≃ \{ \begin{matrix}0 , & if & {C}_{0} > {B}_{max} , \\ \frac{{B}_{max} - {C}_{0}}{μ} , & if & {C}_{0} < {B}_{max} .\end{matrix} \qquad ( 25 )$$

where we introduce the simplifying notation

Bₘₐₓ≡B_{e,max}+B_{i1,max}+B_{i2,max} (26)

As long as Tᵣᵢₛₑ>>T_{y}, Eqs. (24)-(25) imply that

$${C}_{e} ≃ {C}_{e , ss} ≡ \frac{μ}{γ} , t > {T}_{rise} . \qquad ( 27 )$$

From Equations (24) to (27) one notes:

$$\begin{matrix}{T}_{rise} ∝ \frac{{T}_{max}}{{W}_{load}} \\ and & ( 28 ) \\ {C}_{e , ss} ∝ \frac{{W}_{load}}{{T}_{max}} . & ( 29 )\end{matrix}$$

This leads to the result that the rise time, Tᵣᵢₛₑ, and steady state extracellular concentration, C_{e,ss}, are both controllable quantities.

The following illustrations and calculations use the default values for the parameters in Table 2.

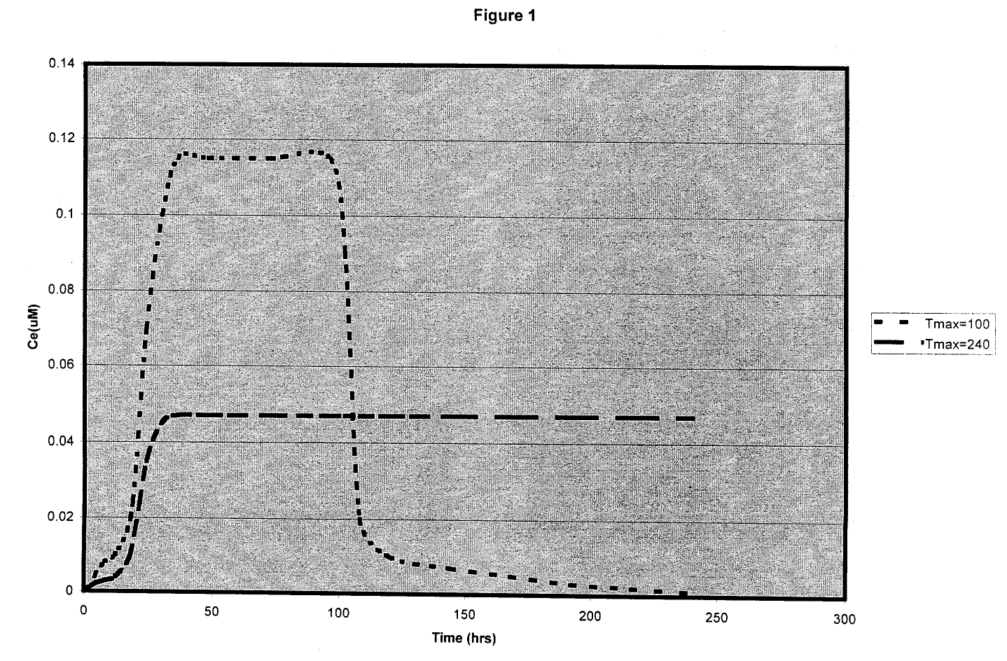

FIG. 1 shows that, when flux of the poorly water soluble antineoplastic drug is zero order, steady state extracellular concentration is proportional to Tₘₐₓ, rise time and steady state concentration are inversely proportional to Tₘₐₓ (see Equations 24 & 25).

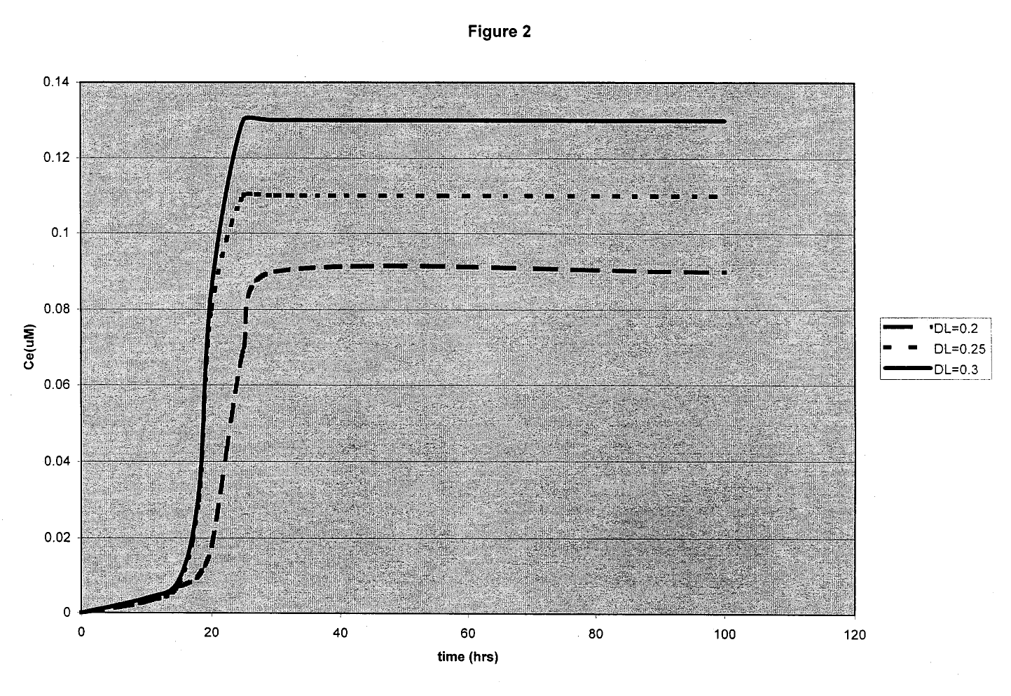

FIG. 2 depicts the extracellular drug concentration profile at different loadings of poorly water soluble antineoplastic drug in the microparticles. Drug loading of course affects the steady-state extracellular concentration and also has an affect on rise time, consistent with equations (24) to (27).

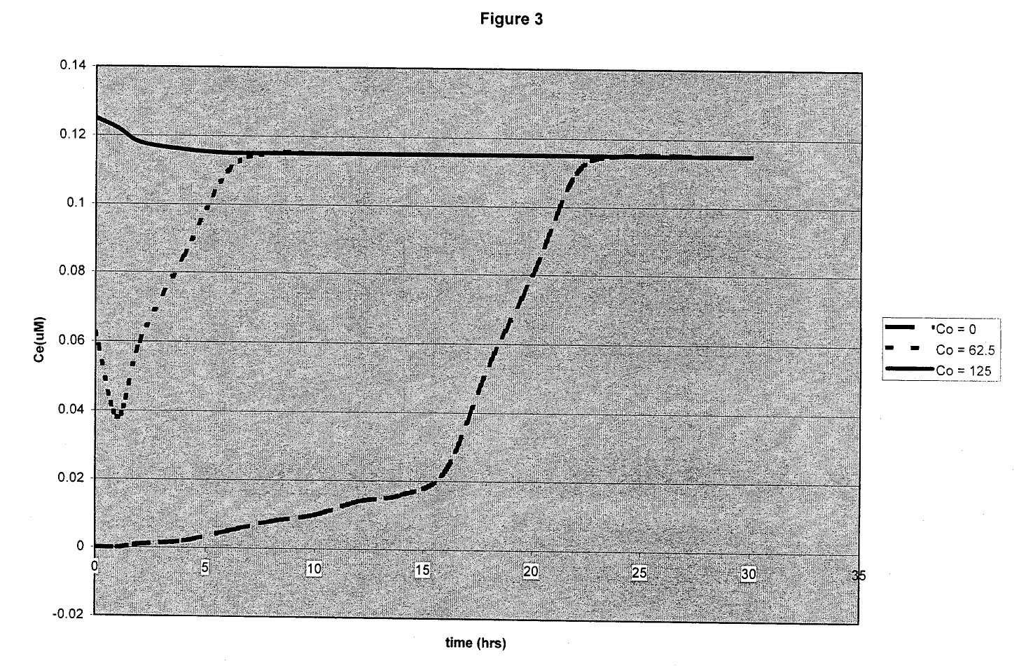

FIG. 3 depicts the effect of an initial free extracellular drug concentration on the concentration vs. time profiles using the default parameters of Table 2.

In conclusion, the present inventors have developed a reaction diffusion model that describes the dynamics of drug release from microspheres injected into solid tumors.

The parameters of this model are measurable quantities with clear physical meaning. The relevant parameter range for paclitaxel release can be estimated from the literature. Zero order release was shown to guarantee an above threshold steady state extracellular concentration of the poorly water soluble antineoplastic drug paclitaxel for a long period of time. The steady state extracellular concentration, C_{e,ss}, is proportional to W_{load}/(Tₘₐₓ) and can therefore be controlled by varying the drug load (W_{load}) and the duration of drug release from the microspheres (Tₘₐₓ). A long duration of drug release leads to a low C_{e,ss}, while a high drug load leads to a high C_{e,ss}.

Furthermore, the maximum duration of the steady state concentration is approximately equal to the duration of drug release from the microspheres, Tₘₐₓ. Due to cellular uptake, the duration of the steady state is shorter than the duration of drug release, Tₛₛ≈Tₘₐₓ−Tᵣᵢₛₑ. This is a problem only if the drug load is low and/or the clearance rate is high, and can be overcome by injecting a loading dose of TaxAlbin® along with the microspheres.

Consistent with the present invention, the model would predict the optimal treatment could be achieved by the injection of 300 mg of microspheres with an average radius of 1.5μ and at least a 20% drug load and with a duration of release of 100 hours. A higher drug load will give a more efficacious drug concentration over the optimum periods. A significantly longer duration of release, e.g. 500 hours, will give a lower concentration and less than optimum results.

The following references are cited above in the discussion of theoretical considerations:

[1] R. K. Jain, J. M. Weisbrod, and J. Wei. Mass transport in tumors: Characterization and applications to chemotherapy. Adv. Cancer Res., 33:251-310, 1980.

[2] X. Y. Wu, G. Eshun, and Y. Zhou. Effect of interparticulate interaction on release kinetics of microsphere ensembles. J. Pharm. Sci., 87:586-593, 1998.

[3] L. T. Baxter, F. Yuan, and R. K. Jain. Pharmacokinetic analysis of the perivascular distribution of bifunctional antibodies and haptens: Comparison with experimental data. Cancer Res., 52:5838-5844, 1992.

[4] L. T. Baxter and R. K. Jain. Pharmacokinetic analysis of the microscopic distribution of enzyme conjugated antibodies and prodrugs: Comparison with experimental data. Br. J. Cancer, 73:447-456, 1996.

[5] H. J. Kuh, S. H. Jang, M. G Wientjes, and J. L. S. Au. Computational model of intracellular pharmacokinetics of paclitaxel. J. Pharm. Exp. Ther., 293:761-770, 2000.

[6] S. H. Jang, M. G. Wientjes, and J. L. S. Au. Kinetics and mathematical modeling of paclitaxel efflux by P-glycoprotein in BC19 cells. Proc. Am. Assoc. Cancer Res., 39:218, 1998.

[7] A. W. El-Kareh and T. W. Secomb. Theoretical models for drug delivery to solid tumors. Crit Rev Biomed Eng., 25:503-571, 1997.

[8] Y. Wang, H. Sato, and I. Horikoshi. In vitro and in vivo evaluation of Taxol release from poly(lactic-co-glycolic acid) microspheres containing isopropyl myristate and degradation of the microspheres. J. Control. Rel., 49:157-166, 1997.

[9] P. Xia, P. M. Bungay, C. C. Gibson, O. N. Kovbasnjuk, and K. R. Spring. Diffusion coefficients in the lateral intercellular spaces of madrine-darby canine kidney cell epithelium determined with caged compounds. Biophys. J., 74:3302-3312, 1998.

[10] A. W. El-Kareh, S. L. Braunstein, and T. W. Secomb. Effect of cell arrangement and interstitial volume fraction on the diffusivity of monoclonal antibodies in tissue. Biophys. J., 64:1638-1646, 1993.

[11] R. K. Jain. Transport of molecules in tumor interstitum: a review. Cancer Res., 47:3039-3051, 1987.

[12] J. Lankelma, R. F. Luque, H. Dekker, W. Schinkel, and H. M. Pinedo. A mathematical model of drug transport in human breast cancer. Microvasc. Res., 59:149-161, 1999.

[13] A. B. Dhanikula and R. Pachagnula. Localized paclitaxel delivery. Int. J. Pharm., 183:85-100, 1999.

[14] M. R. Wenk, A. Fahr, R. Reszka, and J. Seelig. Paclitaxel partitioning, into lipid bilayers. J. Pharm. Sci., 85:228-231, 1996.

[15] M. Nishikawa and M. Hashida. Pharmacokinetics of anticancer drugs, plasmid DNA, and their delivery systems in tissue isolated perfused tumors. Adv. Drug Del. Rev., 40:19-37, 1999.

[16] L. T. Baxter and R. K. Jain. Transport of fluid and macromolecules in tumors. I. Role of interstitial pressure and convection. Microvasc. Res., 37:77-104, 1989.

[17] M. Caplow, J. Shanks, and R. Ruhlen. How Taxol modulates microtuble assembly. J. Biol. Chem., 269:23399-23402, 1994.

[18] Song D., L. F. Hsu, and J. L. S. Au. Binding of Taxol to plastic and glass containers and protein under in vitro conditions. J. Pharm. Sci., 85:29-31, 1996.

[19] R. T. Liggins, W. L. Hunter, and H. M. Burt. Solid-state characterization of paclitaxel. J. Pharm. Sci., 86:1458-1463, 1997.

[20] J. L. S. Au, D. Li, Y. Gan, X. Gao, A. L. Johnson, J. Johnston, N. J. Millenaugh, S. H. Jang, H. J. Kuh, C. T. Chen, and M. G. Wientjes. Pharmacodynamics of immediate and delayed effects of paclitaxel: Apoptosis and intracellular drug retention. Cancer Res., 58:2141-2148, 1998.

[21] J. F. Diaz, R. Strobe, Y. Engleborghs, A. A. Souto, and J. .M. Andreu, Molecular Recognition of Taxol by Microtubules, J. Biol. Chem., 275: 26265-27276 (2000).

### DETAILED DESCRIPTION OF THE INVENTION

In one embodiment, the present invention provides a pharmaceutical powder that includes a poorly water soluble antineoplastic agent and that can be constituted to a pharmaceutical composition suitable for intratumoral injection. Upon intratumoral injection, the pharmaceutical composition of the present invention forms a reservoir from which the poorly water soluble antineoplastic agent is released in a therapeutically effective, extended, and hitherto unachievable time-dependent manner. The method of the present invention results in a more effective intratumoral concentration of the antineoplastic agent. Therapeutic effectiveness can be demonstrated by, for example, tumor growth rate (i.e. size as a function of time), tumor viability, and necrosis, to mention just three, all of which are known in the art.

The pharmaceutical powder of the present invention includes microparticles. The microparticles can have any morphology or construction (e.g. hollow, solid, layered, etc.). The microparticles are constituted of, among other things, a poorly water soluble antineoplastic agent, most preferably paclitaxel, and at least one water soluble polymer. The powder can also contain adjuvants and/or excipients that assist in constitution. Although the present invention is not dependent on a particular theory of operation, it is thought that forming the microparticles with water soluble polymer allows for a more rapid release of the antineoplastic agent. The water soluble polymers enhance the dissolution of the poorly water soluble antineoplastic agent giving the desired release rate.

Intratumoral injection is well known in the medical arts as discused above. In this route of administration a pharmaceutical composition is injected directly into a tumor

Paclitaxel, the active pharmaceutical ingredient in Taxol®, is the preferred antineoplastic agent in the practice of the present invention. Use of paclitaxel in cancer chemotherapy is well known and is discussed above. Any paclitaxel useful in known conventional cancer chemotherapy can be used in the practice of the present invention.

The water soluble polymers useful in the practice of the present invention are well known in the art and include, inter alia, polyvinyl pyrrolidone (PVP), polyvinyl alcohol (PVA), modified celluloses including hydroxypropyl cellulose, methylcellulose, hydroxtpropylmethylcellulose, sodium carboxymethylcellulose, and hydroxyethylcellulose, polysaccharides such as sodium alginate, pectin, chitosan, xanthan gum, carrageenen, guar gum, and gum tragaganth, to mention just a few. Polyvinyl pyrrolidone (PVP) is the preferred water soluble polymer in the practice of the present invention.

In addition to the poorly water soluble antineoplastic agent and water soluble polymer, the microparticles used in the practice of the present invention can also include adjuvants, excipients, or both. The excipients can be emulsifiers or surface active agents, to mention just two. Examples of these excipients include the polysorbates, the ethoxylate sorbitans, and phospholipids.

In the following discussion, it will be understood that mention of sizes, dimensions, or weights of microparticles does not refer to a particular isolate microparticle, but rather to the nominal average size, dimension, or weight for a statistically significant sample of particles such as may be contained in an aliquot of the pharmaceutical powder of the present invention.

Preferred microparticles of the present invention have at least about 50% and as much as about 90% by weight antineoplastic agent, preferably paclitaxel, the remainder being water soluble polymer, preferably PVP, and excipients and adjuvants, if any.

Particularly preferred microparticles have between about 65% by weight and about 75% by weight of the microparticles paclitaxel, the remainder being water soluble polymer, preferably PVP and, optionally, excipients, adjuvants, or both.

The microparticles of the pharmaceutical powder of the present invention have an average nominal diameter between about 0.5μ and about 10μ. In preferred embodiments, the microparticles have an average nominal diameter between about 1μ and about 5μ. In a particularly preferred embodiment, the microparticles have and average nominal diameter between about 2μ and about 4μ.

It will be understood that reference to average diameter of a particle does not refer to any particular individual particle but rather to the average nominal diameter of a statistically significant sample of particles.

The microparticles can be prepared using techniques well-known in the art. For example, they can be prepared by the so-called solvent evaporation technique. See Liggins, R. T. and Burt, H., Paclitaxel loaded poly(L-lactic acid) microspheres: Properties of microspheres made with low molecular weight polymers, Int. J. Pharm. 2001, 222(1), 19-33; Liggins, R. T., et. al., Paclitaxel loaded poly(L-lactic acid) microspheres for the prevention of intraperitoneal carcinomatosis after a surgical repair and tumor cell spill, Biomaterials, 2000, 21(19), 1959-1969, all of which are incorporated herein by reference in their entirety. See also Burt, H. M., et. al. , Controlled delivery of Taxol from microspheres composed of a blend of ethylene-vinyl acetate copolymer and poly(d,l lactic acid), Cancer Lett. 1995, 88(1), 73-79), incorporated herein in its entirety by reference.

The microparticles can also be prepared by the so-called solvent extraction technique. See, e.g., Feng, S. and Huang, G. , Effects of emulsifiers on the controlled release of paclitaxel (Taxol) from nanospheres of biodegradable polymers, J. Control. Release 2001 , 71(1), 53-59 ; Shiga, K. et. al. , Preparation of poly(d,l-lactide) and copoly(lactide-glycolide) microspheres of uniform size, J. Pharm. Pharmacol. 1996, 48 (9), 891-5; both of which are incorporated herein in their entirety by reference. See also Schaefer, M. J. and Singh, J. , Effects of additives on stability of etoposide in PLGA microspheres, Drug Dev. Ind. Pharm. 2001, 27 (4), 345-350), incorporated herein in its entirety by reference.

In either the solvent evaporation technique or the solvent extraction technique, the poorly water soluble antineoplastic agent, preferably paclitaxel, and water soluble polymer are dissolved in a suitable organic solvent that is partly miscible with water such as dichloromethane or ethyl acetate. A water solution of either polyvinyl alcohol or gelatin (to aid in emulsification) is added to the solution and the mixture emulsified using either high speed stirring (using a high speed, high shear mixer such as a Silverson homogenizer or the like) or ultrasonic energy. The size of the emulsified organic droplets is dependent on the speed of mixing or the energy of the ultrasound irradiation, the concentration of the components in each phase, and the ratio of the volumes of the organic and water phases. In general, the higher the speed of mixing or energy of irradiation, the more concentrated the solution and the higher the water-to-organic solvent ratio, the smaller the droplets. One skilled in the art knows how to manipulate these parameters by routine experimentation to obtain the desired microparticle size. The emulsified droplets are converted to microparticles by removing the organic solvent either by raising the temperature and causing evaporation while stirring (solvent evaporation technique) or by extracting the organic solvent out of the droplets with another solvent (solvent extraction technique).

In the solvent extraction technique, the extracting solvent can be another organic solvent in which the components of the microparticle are not very soluble, or a large volume of cooled water (large enough to dissolve the organic solvent which is poorly soluble in water, but not enough to dissolve the water soluble polymer in the microparticle). The formed microparticles are collected by either filtration or centrifugation.

Most of the prior art deals with microparticles based on polymers and co-polymers that are not water soluble such as polylactide and polylactide-co-glycolide. The polymer slows drug release, releasing the drug by diffusion through the matrix and by erosion of the matrix. In such cases the rate of drug release is controlled by the particle size (which controls surface area), the porosity built into the microparticles, additives such as emulsifiers which can be added to the emulsification step, and the rate of degradation of the microparticles which is mostly controlled by the type of polymer used and its molecular weight. The present invention does not use a polymer to slow down the drug release. Paclitaxel is an example of a poorly water soluble antineoplastic agent and its release from neat paclitaxel particles is too slow in vivo to be effective in intratumoral injection. While not bound to any theory of operation, it is thought that the water soluble polymers used in the practice of the present invention speed-up the drug release from the microparticles.

The rate of release of the drug from the microparticles particles can be controlled by controlling, among other things, the particle size, the water soluble polymer used in making the microparticle, the percent of the polymer in the particle, and the molecular weight of the polymer. The greater the water solubility of the water soluble polymer, the faster will be the release of the poorly water soluble antineoplastic agent. The higher the weight percent of the water soluble polymer, the higher will be the rate of release of the poorly water soluble antineoplastic agent. The higher the molecular weight of the polymer the slower the polymer dissolves, thereby slowing down the release rate of the poorly water soluble antineoplastic agent. One can, optionally, also add soluble small molecules as excipient to aid in the dissolution of the antineoplastic agent. Excipients useful for this purpose include water soluble salts, low molecular weight sugars, surface active agents, and emulsifiers. Examples of such salts include sodium or potassium chloride or nitrate, to mention just a few. Examples of such sugars include sucrose, glucose, fructose, sorbitol, and maltose, to mention just a few.

The pharamaceutical powder can be comprised of microparticles alone, or the microparticles can be combined with additional excipients or adjuvants.

For use in injection, especially intratumoral injection, the pharmaceutical powder of the present invention is constituted with an injection vehicle and, if desired, one or more adjuvants, for example an isotonic agent, or excipients, for example a preservative or suspending aid, to the injectable pharmaceutical composition that is another embodiment of the present invention.

The injection vehicle can be any injection vehicle known in the art; for example aqueous vehicles, water-miscible vehicles, and nonaqueous vehicles. Water is the preferred injection vehicle in the practice of the present invention. It will be understood that water refers to water for injection (WFI). The pharmaceutical powder is combined with and suspended in the injection vehicle at a concentration between about 20 and about 400 mg/ml, preferably between about 200 and about 300 mg/ml, in a suitable container (e.g. vial or test tube that can be sealed with a serum stopper). Agitation required to effect suspension can be effected with any device known in the art, for example a high speed orbital-type mixer.

An example of an injection vehicle is a solution of 0.5% (w/v) of low-viscosity sodium carboxymethylcelloulose as a suspension aid, 0.1% (w/v) Tween® 20, the remainder being 0.9% (w/v) NaCl in water for injection.

Isotonizing agents are well known in the art and are examples of adjuvants that can be used in making the pharmaceutical compositions of the present invention. Other antineoplastic agents, including a solubilized form paclitaxel itself, can be used as adjuvants

If needed or desired, excipients can also be included in the pharmaceutical composition. Buffers and antimicrobals are just two examples of useful excipients.

In another embodiment, the present invention provides a method of treating a solid tumor in a mammal, preferably a human, with the pharmaceutical composition of the present invention which contains microparticles of the present invention that are small in size and highly loaded with an antineoplastic agent, preferably paclitaxel. In this embodiment, the pharmaceutical composition is injected to form a depot or reservoir. The injection can be subcutaneous, intramuscular, or intratumoral. In particularly preferred embodiments, the injection is intratumoral.

As discussed above, the technique of intratumoral injection is generally known to practitioners in the medical arts. The amount of pharmaceutical composition injected is between about 5 vol-% and about 25 vol-% of the volume of the tumor to be treated. If the tumor weight is about 2 g and the concentration of microspheres in the pharmaceutical composition is about 250 mg of particles per mL of pharmaceutical composition; about 125 mg of microparticles will be delivered. In preferred embodiments, the loading of antineoplastic agent in the microspheres and the concentration of the pharmaceutical composition are adjusted so that at least about 8 mg of antineoplastic agent are delivered per gram of tumor weight, preferably 30 mg to 50 mg per gram of tumor weight.

Upon intratumoral injection, the pharmaceutical particles of the present invention spread throughout the tumor in an approximately homogeneous fashion. The paclitaxel is prefereably released from the particles over a period of 24 to 240 hours, more preferably over a period of 48 to 100 hours.

The pharmaceutical powders and pharmaceutical compositions of the present invention can also be used to form a depot of microspheres for local or systemic drug release by, for example, injecting the composition subcutaneously or intramuscularly.

The present invention can be illustrated by the following non-limiting examples.

### EXAMPLE 1

### Microsphere Spread in a Tumor

The objective of the study was to determine (1) the effect of pre-injection of TaxAlbin® (soluble paclitaxel) on microsphere dispersion within a human adenocarcinoma tumor xenograft and (2) determine effect of microsphere particle size on the extent of microsphere dispersion within a murine tumor. In this study, a dispersion of Fluorescent Commercial Microspheres (Placebo) was administered following injection of TaxAlbin® 24 hours prior to injection of the microspheres.

The microspheres used in this study were Fluoresbrite plain YG 2.0 micron and 10.0 micron obtained from Polysciences Europe GmbH.

Twelve nude mice injected with xenograft tumor (MCF7 human breast adenocarcinoma) were the animal models in this study. Mice were inoculated with 10⁷/0.1 ml human mammary tumor cell line MCF7. Tumors were allowed to grow for 4 weeks to reach approximate size of 1-2 grams.

Each of the mice received two injections within 24 hours. The first was either TaxAlbin® or saline, and the second, at 24 hours, was commercial fluorescent microspheres of either 2μ or 10 μm particle size. Thus, the following 4 treatments were evaluated:

TaxAlbin® injection+microsphere (2 microns) injection

TaxAlbin® injection+microsphere (10 microns) injection

Saline injection+microsphere (2 microns) injection

Saline injection+microsphere (10 microns) injection

Tumors were excised from the mice and cut open in two orthogonal directions. Opening up the tumor to see all the cut surfaces gives a view on the spread of the microspheres in each direction. The tumors were then viewed under UV light and the homogeneity of the microspheres' spread accessed qualitatively.

The extent of microsphere dispersion was evaluated by presence of fluorescent dye.

The results of the qualitative assessment of the tumors are summarized in Table 3.

TABLE 3

| | w/saline preinjection | w/TaxAlbin ® preinjection |
|---|---|---|
| 2 micron diameter | apparent homogeneous | apparent homogeneous |
| | spread | spread |
| 10 micron diameter | spread to majority of | apparent almost |
| | tumor | homogeneous spread |

The smaller (2μ) microspheres were homogeneously spread throughout the tumor without any pretreatment. The larger (10μ) microspheres spread through most of the tumor, but there were areas where they were apparently absent. Pretreatment with TaxAlbin® improved the spread of the larger microspheres.

### EXAMPLE 2

### Mouse Xenograft Trial

Effects of Administration of Paclitaxel Microparticlels on a Subcutaneously Implanted Human Breast Xenograft

The human breast tumor cell line MCF7 (ECACC, estrogen-independent variant) is maintained in serial passage in female immunodeficient mice (Cancer Studies Unit, University of Nottingham). To set up the studies, tumor from donor animals was excised, removed from the capsule, pooled and finely minced. Pieces ca. 3 mm³ each were implanted, under anesthetic (Hypnorm, Roche/Hypnovel, Jansen), subcutaneously, into the left flank of female MF 1 nude mice (Cancer Studies Unit, University of Nottingham). The mice were electronically tagged (Trovan, R. S. Biotech) and assigned to the relevant experimental groups. Tumors were measured 3 times weekly from day 7, and dosing was carried out when the group mean cross-sectional area, measured in two perpendicular dimensions, reached ˜50 mm² (approx. day 14/15). The treatment groups were designed to test the paclitaxel microspheres using several protocols. Group 3 tested the efficacy of the microspheres themselves with no pretreatment and with no loading dose of a soluble paclitaxel solution. Group 2 had the microspheres suspended in a soluble paclitaxel solution whilst in Group 4, the microspheres were suspended in the soluble paclitaxel and models were given a pretreatment of the soluble paclitaxel 24 hours before dosing with the microparticles. Group 2 was designed to test whether a loading dose of soluble drug offers a therapeutic advantage when compared to release from the microspheres alone. Group 4 tested whether there a further advantage of pretreating the tumor with a soluble paclitaxel could be observed.

Such pretreatment has been reported to cause apoptosis and may aid the subsequent spread of the microsphere treatment. Paclitaxel solublized in 20% human serum albumin (TaxAlbin®) was used as the soluble paclitaxel.

For the study, 42 female nude mice were initiated as above and allocated to the following dosing groups.

Group 1 Treatment 1 (Day 0): Intratumoral injection of 50 μl TaxAlbin®; n=8 mice

Treatment 2 (Day 1): Intratumoral injection of 50 μl TaxAlbin®

Treatment 3 (Day 2): Intratumoral injection of 50 μl TaxAlbin®

Group 2 Treatment 1 (Day 0): Intratumoral injection of 50 μl paclitaxel/PVP; n=8 mice. Particles suspended in TaxAlbin®

Group 3 Treatment 1 (Day 0): Intratumoral injection of 50 μl paclitaxel/PVP;n=8 mice. Particles suspended in injection vehicle.

Group 4 Treatment 1 (Day 0): Intratumoral injection of 50 μl TaxAlbin®; n=8 mice.

Treatment 2 (Day 1): Intratumoral injection of paclitaxel/PVP particles suspended in TaxAlbin®.

Group 5 Untreated (control); n=5 mice.

Group 6 Treatment 1 (Day 0): Intratumoral injection of 50 μl saline; n=5 mice.

Treatment 2 (Day 1): Intratumoral injection of 50 μl saline.

Treatment 3 (Day 2): Intratumoral injection of 50 μl saline.

The mice were terminated on day 19 following injection. At the end of the study, the DNA analogue, bromodeoxyuridine, was administered (160 mg/kg), 1 hour prior to termination, to allow determination of proliferation within the tumor.

Tumors were dissected out and weighted. Tumor samples were snap frozen and stored for further analysis, as required. Additionally, samples were fixed in formalin and processed to paraffin for histological analysis. The latter were required to ascertain the degree of necrosis within the tumor together with evaluation of the degree of mechanical disruption caused by the intratumoral injection.

Haematoxylin and Eosin stained sections through subcutaneous tumors were taken at study termination.

The studies conformed to the United Kingdom Co-Coordinating Committee on Cancer Research (UKCCCR) Guidelines.

### Description of Materials Used in the Study

TaxAlbin®, when reconstituted, is a solution of paclitaxel at a concentration of 1 mg/ml in 20% human serum albumin.

Paclitaxel/PVP microparticles are particles that contain 75% paclitaxel and 25% PVP with an average particle size of 3.5 micron. The microparticles were prepared as described below.

Paclitaxel, 160 mg, was dissolved in 3 mL dichloromethane. Polyvinylpyrrolidone, 70 mg, was added and the solution was stirred until all had dissolved. Twelve milliliters of a water solution of polyvinylalcohol (2 weight percent ) were added. The mixture was then emulsified for 4 minutes at about 9000 rpm using a Silverson homogenizer. The emulsion thus formed was poured into 170 mL of ultrapure water pre-chilled in an ice-water bath. The microparticles were collected by centrifugation, resuspended in one milliliter water, 0.2 ml of 15% w/v mannitol solution was added and the suspension lyophilized. The obtained microparticles were analyzed by HPLC for paclitaxel content, by laser light scattering for particle size, and by optical microscope for morphology. The results are in Table 4.

TABLE 4

| Property | Result |
|---|---|
| paclitaxel content | 74.9% w/w |
| median diameter [D(V, 0.5)] | 3.47μ |
| particle size distribution | 1.47-6.89μ |
| [D(V, 0.1)-D(V, 0.9)] | |
| morphology | small microparticles, no aggregates, |
| | no free crystals of paclitaxel |

TABLE 5
Treatment schedule for CSU Trial in a mouse breast
xenograft tumor model

| Group | Group | Treatment 1 | Treatment 2 | Treatment 3 |
|---|---|---|---|---|
| No. | size | on Day 0 | on Day 1 | on Day 2 |
| 1 | 8 | intratumoral | intratumoral | intratumoral |
| | | injection of: | injection of: | injection of: |
| | | 50 μl | 50 μl | 50 μl |
| | | TaxAlbin ® | TaxAlbin ® | TaxAlbin ® |
| 2 | 8 | intratumoral | N/A | N/A |
| | | injection of: | | |
| | | 50 μl | | |
| | | suspension of | | |
| | | paclitaxel/PVP | | |
| | | particles in | | |
| | | TaxAlbin ® | | |
| 3 | 8 | intratumoral | N/A | N/A |
| | | injection of: | | |
| | | 50 μl | | |
| | | suspension of | | |
| | | paclitaxel/PVP | | |
| | | particles in | | |
| | | injection | | |
| | | diluent | | |
| 4 | 8 | intratumoral | intratumoral | N/A |
| | | injection of: | injection of: | |
| | | 50 μl | 50 μl | |
| | | TaxAlbin ® | suspension of | |
| | | | paclitaxel/PVP | |
| | | | particles in | |
| | | | TaxAlbin ® | |
| 5 | 5 | N/A | N/A | N/A |
| | | (Untreated | (Untreated | (Untreated |
| | | control) | control) | control) |
| 6 | 5 | intratumoral | intratumoral | intratumoral |
| | | injection of: | injection of: | injection of: |
| | | 50 μl saline | 50 μl saline | 50 μl saline |

TABLE 6
Paclitaxel dosages for administration in a mouse
breast xenograft tumor model

| | | Paclitaxel |
|---|---|---|
| Group No. | Treatments | dosages per mouse |
| 1 | 50 μl TaxAlbin ® on Days 0, 1 and 2 | 0.05 mg per |
| | | administration |
| 2 | 50 μl suspension of paclitaxel/PVP | 2.25 mg comprising: |
| | particles in TaxAlbin ® on Day 0 | 2.2 mg from |
| | | paclitaxel/PVP |
| | | particles + 0.05 mg |
| | | from TaxAlbin ® |
| 3 | 50 μl suspension of paclitaxel/PVP | 2.25 mg from |
| | particles in injection diluent on | paclitaxel/PVP |
| | Day 0 | particles |
| 4 | 50 μl TaxAlbin ® on Day 0 | 0.05 mg on Day 0 |
| | 50 μl suspension of paclitaxel/PVP | 2.25 mg on Day 1 |
| | particles in TaxAlbin ® on Day 1 | comprising: 2.2 mg |
| | | from paclitaxel/PVP |
| | | particles + 0.05 mg |
| | | from TaxAlbin ® |
| 5 | Untreated control | 0 |
| 6 | 50 μl saline on Days 0, 1 and 2 | 0 |

### Results

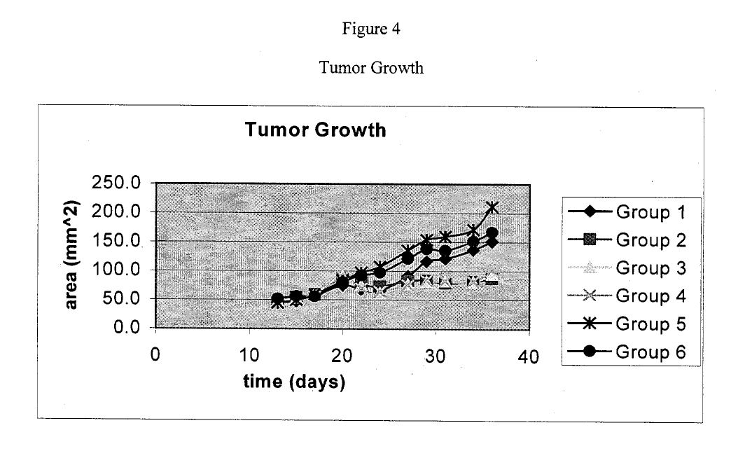

The result of the average measurements of the crossectional area of the tumors as a function of time are given in Table 7 and shown graphically in FIG. 4. The results of the tumor wieghts at trial end are given in Table 8.

TABLE 7
Crossectional Area of Tumors (mm²)

| | | | inject | | | | | | | | |
|---|---|---|---|---|---|---|---|---|---|---|---|
| | DAY | DAY | DAY | DAY | DAY | DAY | DAY | DAY | DAY | DAY | DAY |
| | 13 | 15 | 17 | 20 | 22 | 24 | 27 | 29 | 31 | 34 | 36 |
| Group 1 | | | | | | | | | | | |
| MEAN | 45.4 | 46.9 | 56.5 | 74.8 | 68.3 | 65.6 | 91.3 | 116.7 | 121.3 | 136.8 | 151.2 |
| ST.DEV | 10.8 | 9.9 | 13.3 | 16.6 | 28.1 | 27.8 | 46.8 | 57.5 | 60.5 | 69.1 | 73.6 |
| median | 44.4 | 42.8 | 55.4 | 75.6 | 66.8 | 70.3 | 112.7 | 150.7 | 153.0 | 158.4 | 180.9 |
| Group 2 | | | | | | | | | | | |
| MEAN | 46.1 | 54.5 | 60.2 | 87.9 | 76.4 | 73.6 | 82.9 | 83.7 | 78.9 | 82.3 | 87.1 |
| ST.DEV | 11.1 | 18.4 | 22.6 | 20.2 | 26.3 | 28.8 | 37.9 | 44.1 | 50.0 | 57.7 | 60.3 |
| median | 45.4 | 51.2 | 51.5 | 86.5 | 69.5 | 66.6 | 74.5 | 68.1 | 63.4 | 60.8 | 70.1 |
| Group 3 | | | | | | | | | | | |
| MEAN. | 47.1 | 52.8 | 60.0 | 91.1 | 75.2 | 62.0 | 77.0 | 81.7 | 82.7 | 81.3 | 95.3 |
| ST.DEV | 13.1 | 9.1 | 13.7 | 14.8 | 23.8 | 9.7 | 33.5 | 45.5 | 45.6 | 55.4 | 65.6 |
| median | 44.5 | 51.5 | 57.3 | 84.0 | 66.9 | 63.7 | 67.0 | 64.0 | 68.5 | 58.5 | 74.6 |
| Group 4 | | | | | | | | | | | |
| MEAN | 44.9 | 48.7 | 55.2 | 90.1 | 69.7 | 64.5 | 75.8 | 83.9 | 83.3 | 82.3 | 101.2 |
| ST.DEV | 12.8 | 16.1 | 18.5 | 25.4 | 22.7 | 17.2 | 29.8 | 35.2 | 30.0 | 34.0 | 43.3 |
| median | 39.7 | 44.2 | 47.8 | 80.5 | 70.6 | 60.2 | 67.4 | 85.0 | 80.4 | 86.3 | 113.0 |
| Group 5 | | | | | | | | | | | |
| MEAN. | 44.6 | 49.2 | 59.2 | 82.8 | 97.4 | 107.3 | 135.6 | 153.9 | 159.6 | 171.5 | 211.2 |
| ST.DEV | 11.2 | 12.1 | 15.6 | 23.1 | 30.6 | 39.8 | 44.2 | 49.0 | 49.9 | 52.5 | 56.7 |
| Median | 50.4 | 49.5 | 60.0 | 88.7 | 103.0 | 123.9 | 160.1 | 179.0 | 182.1 | 199.1 | 227.8 |
| Group 6 | | | | | | | | | | | |
| MEAN | 51.9 | 55.9 | 55.5 | 80.7 | 92.7 | 97.2 | 121.2 | 138.4 | 135.5 | 151.6 | 167.1 |
| ST.DEV | 19.6 | 19.5 | 60.7 | 32.9 | 44.2 | 44.5 | 56.5 | 66.2 | 63.1 | 70.7 | 75.1 |
| Median | 52.8 | 60.0 | 66.0 | 94.8 | 109.9 | 116.1 | 140.6 | 153.9 | 155.4 | 181.9 | 192.7 |

TABLE 8
Final Weights(grams) of Excised Tumors

| | GROUP 1 | GROUP 2 | GROUP 3 | GROUP 4 | GROUP 5 | GROUP 6 |
|---|---|---|---|---|---|---|
| | TUMOR | TUMOR | TUMOR | TUMOR | TUMOR | TUMOR |
| | WGT(gm) | WGT(gm) | WGT(gm) | WGT(gm) | WGT(gm) | WGT(gm) |
| | 0.09 | 0.03 | 0.14 | 0.02 | 0.39 | 0.02 |
| | 0.45 | 0.03 | 0.06 | 0.12 | 0.76 | 0.5 |
| | 0.74 | 1.26 | 0.32 | 0.49 | 1.25 | 0.73 |
| | 0.80 | 0.18 | 1.39 | 0.22 | 1.05 | 1.54 |
| | 1.01 | 0.07 | 0.07 | 0.49 | 1.17 | 0.76 |
| | 0.03 | 0.44 | 0.07 | 0.29 | — | — |
| | — | 0.09 | 0.06 | 0.27 | — | — |
| | 0.60 | 0.08 | 0.09 | 0.14 | — | — |
| MEAN | 0.53 | 0.27 | 0.275 | 0.26 | 0.92 | 0.71 |
| ST.DEV | 0.34 | 0.39 | 0.43 | 0.16 | 0.31 | 0.49 |
| MEDIAN | 0.60 | 0.09 | 0.08 | 0.25 | 0.92 | 0.73 |

One can clearly see that the treatment groups 2, 3,and 4, when compared to the no treatment and sham treatment groups, had smaller tumors both in cross-sectional area, (70.1, 74.6, 113.0, vs. 227.8, 192.7 median area in mm², respectively) and in final tumor weight (0.09, 0.08, 0.25 vs. 0.92, 0.73 median weight in grams, respectively). No advantage was seen for either a loading dose of soluble paclitaxel nor for a pretreatment with a soluble paclitaxel. Group 1 showed an initial effect in retarding tumor growth. The rate of tumor growth recovered in Group 1 by day 27.

The viability of the residual tumors was tested on slices of the excised tumor by The individual results of tumor weight, percent necrosis and percent proliferation at trial end are given in Table 9. Also in Table 9 are the calculated weight of the tumor in grams that is non-necrotic and that is proliferating.

TABLE 9
Tumor Weight, Necrosis and Proliferation at Trial End

| | | | | wgt non | wgt |
|---|---|---|---|---|---|
| mouse id | wgt (gm) | % necrosis | % prolif | necrotic | prolif |
| group 1 | | | | | |
| 1 | 0.09 | 17.83 | 14.20 | 0.07 | 0.01 |
| 2 | 0.45 | 37.80 | 19.20 | 0.28 | 0.09 |
| 5 | 0.80 | 67.88 | 16.90 | 0.26 | 0.14 |
| 10 | 0.74 | 61.97 | 7.61 | 0.28 | 0.06 |
| 13 | 0.60 | 43.09 | 13.60 | 0.34 | 0.08 |
| 37 | 1.01 | 72.20 | 13.70 | 0.28 | 0.14 |
| 43 | | | | | |
| 44 | 0.03 | 16.21 | 14.50 | 0.03 | 0.00 |
| mean | 0.53 | 45.28 | 14.24 | 0.22 | 0.07 |
| median | 0.60 | 43.09 | 14.20 | 0.28 | 0.08 |
| group 2 | | | | | |
| 7 | 0.03 | 99.34 | 0.00 | 0.00 | 0.00 |
| 1R | 0.07 | 88.00 | 3.57 | 0.01 | 0.00 |
| 14 | 0.08 | 12.62 | 3.69 | 0.07 | 0.00 |
| 17 | 0.03 | 100.00 | 0.00 | 0.00 | 0.00 |
| 21 | 1.26 | 42.24 | 14.30 | 0.73 | 0.18 |
| 27 | 0.09 | 9.73 | 10.70 | 0.08 | 0.01 |
| 36 | 0.18 | 72.45 | 2.24 | 0.05 | 0.00 |
| 41 | 0.44 | 41.75 | 4.54 | 0.26 | 0.02 |
| mean | 0.27 | 58.27 | 4.88 | 0.15 | 0.03 |
| median | 0.09 | 57.35 | 3.63 | 0.06 | 0.00 |
| group 3 | | | | | |
| 8 | 1.39 | 47.15 | 17.00 | 0.73 | 0.24 |
| 16 | 0.06 | 80.45 | 0.00 | 0.01 | 0.00 |
| 20 | 0.09 | 99.25 | 0.00 | 0.00 | 0.00 |
| 22 | 0.06 | 95.46 | 0.00 | 0.00 | 0.00 |
| 23 | 0.32 | 16.80 | 17.60 | 0.27 | 0.06 |
| 40 | 0.07 | 99.84 | 2.37 | 0.00 | 0.00 |
| 42 | 0.07 | 99.57 | 0.91 | 0.00 | 0.00 |
| 47 | 0.14 | 32.74 | 18.60 | 0.09 | 0.03 |
| mean | 0.28 | 71.41 | 7.06 | 0.14 | 0.04 |
| median | 0.08 | 87.95 | 1.64 | 0.01 | 0.00 |
| group 4 | | | | | |
| 6 | 0.27 | 23.12 | 18.90 | 0.21 | 0.05 |
| 18 | 0.12 | 6.98 | 2.54 | 0.11 | 0.00 |
| 19 | 0.22 | 57.24 | 0.00 | 0.09 | 0.00 |
| 29 | 0.14 | 64.88 | 9.13 | 0.05 | 0.01 |
| 32 | 0.49 | 35.52 | 17.90 | 0.32 | 0.09 |
| 33 | 0.29 | 3.54 | 4.15 | 0.28 | 0.01 |
| 39 | 0.49 | 52.91 | 12.60 | 0.23 | 0.06 |
| 48 | 0.02 | 98.55 | 0.00 | 0.00 | 0.00 |
| mean | 0.26 | 42.84 | 8.15 | 0.16 | 0.03 |
| median | 0.25 | 44.22 | 6.64 | 0.16 | 0.01 |
| group 5 | | | | | |
| 4 | 0.39 | 0.11 | 17.50 | 0.39 | 0.07 |
| 26 | 1.05 | 71.48 | 13.90 | 0.30 | 0.15 |
| 38 | 0.76 | 81.32 | 14.40 | 0.14 | 0.11 |
| 45 | 1.25 | 59.22 | 13.20 | 0.51 | 0.17 |
| 49 | 1.17 | 64.37 | 3.41 | 0.42 | 0.04 |
| mean | 0.92 | 55.30 | 12.48 | 0.35 | 0.11 |
| median | 1.05 | 64.37 | 13.90 | 0.39 | 0.11 |
| group 6 | | | | | |
| 3 | 0.73 | 0.10 | 22.70 | 0.73 | 0.17 |
| 15 | 1.54 | 81.57 | 15.00 | 0.28 | 0.23 |
| 25 | 0.76 | 76.34 | 15.80 | 0.18 | 0.12 |
| 34 | 0.02 | 53.59 | 16.00 | 0.01 | 0.00 |
| 35 | 0.50 | 74.19 | 11.10 | 0.13 | 0.06 |
| mean | 0.71 | 57.16 | 16.12 | 0.27 | 0.12 |
| median | 0.73 | 74.19 | 15.80 | 0.18 | 0.12 |

One can again see that the three treatment groups (groups 2, 3 and 4) clearly had less proliferating tissue than the control groups (0.00, 0.00, and 0.01 vs. 0.11 and 0.12 for the median values respectively) and less non-necrotic tissue than the control groups (0.06, 0.01 and 0.16 vs. 0.39 and 0.18 for the median values respectively). Perhaps the most outstanding of the results is that the treatment groups show many mice with no prolifreration tissue whatsoever. Table 10 collects the results of “non-proliferating tissue” for the variou groups.

TABLE 10
Number of Mice Showing no Proliferating Tissue

| Group | # of mice | # w/no proliferation |
|---|---|---|
| 1 | 7 | 1 |
| 2 | 8 | 5 |
| 3 | 8 | 5 |
| 4 | 8 | 3 |
| 5 | 5 | 0 |
| 6 | 5 | 1 |

In treatment groups 2 and 3 we find 5 of 8 mice with no proliferating tissue while in treatment group 4 we find 3 such mice (and two others that had 0.01 gram of proliferating tissue). In the control groups we find 0 of 5 in the no treatment group and 1 of 5 in the sham treatment group. One may again conclude that all three protocols for the microsphere preparations are efficacious treatments and that neither the loading dose of soluble paclitaxel nor a pretreatment with soluble paclitaxel shows any advantage in the treatment. Group 1 behaves much like the non treated groups at the end of the experiment as would be expected from the data on tumor growth.

What is claimed is:

1. A pharmaceutical powder, capable of being constituted to a pharmaceutical composition for intratumoral injection, comprising microparticles comprising from about 50% by weight to about 90% by weight, based on the weight of the microparticles, of paclitaxel, the remainder by weight of the microparticles comprising at least one water soluble polymer.

2. The pharmaceutical powder of claim 1 wherein the remainder by weight of the microparticles further comprises one or more pharmaceutically acceptable additives selected from emulsifiers and surfactive agents.

3. The pharmaceutical powder of claim 1 wherein the water soluble polymer is selected from the group consisting of polyvinylpyrrolidone, hydroxypropylcellulose, methylcellulose, hydroxypropylmethylcellulose, sodium carboxymethylcellulose, hydroxyethylcellulose, and polysaccharides.

4. The pharmaceutical powder of claim 3 wherein the water soluble polymer is polyvinylpyrrolidone.

5. The pharmaceutical powder of claim 1 wherein the microparticles have an average diameter between about 0.5μ and about 10μ.

6. The pharmaceutical powder of claim 5 wherein the microparticles have an average diameter between about 1μ and about 5μ.

7. The pharmaceutical powder of claim 1 wherein the microparticles have an average diameter between about 2μ and about 4μ and comprise between about 65% by weight and about 75% by weight paclitaxel and between about 25% by weight and about 35% by weight polyvinylpyrrolidone.

8. A pharmaceutical powder, capable of being constituted to a pharmaceutical composition for intratumoral injection, comprising microparticles having an average diameter between about 2μ and about 4μ wherein the microparticles comprise from between about 65% by weight to about 75% by weight, based on the weight of microparticles, of paclitaxel and between about 25% by weight and about 35% by weight, based on the weight of microparticles, of polyvinylpyrrolidone.

9. A pharmaceutical composition, suitable for intratumoral injection, comprising microparticles wherein the microparticles comprise from about 50% by weight to about 90% by weight of paclitaxel, the remainder by weight of the microparticles comprising at least one water soluble polymer.

10. The pharmaceutical composition of claim 9 wherein the remainder by weight of the microparticles further comprises one or more pharmaceutically acceptable additives selected from emulsifiers and surfactive agents.

11. The pharmaceutical composition of claim 9 wherein the water soluble polymer is selected from the group consisting of polyvinylpyrrolidone, hydroxypropylcellulose, methylcellulose, hydroxypropylmethylcellulose, sodium carboxymethylcellulose, hydroxyethylcellulose, and polysaccharides.

12. The pharmaceutical composition of claim 11 wherein the water soluble polymer is polyvinylpyrrolidone.

13. The pharmaceutical composition of claim 9 wherein the microparticles have an average diameter between about 0.5μ and about 10μ.

14. The pharmaceutical composition of claim 13 wherein the microparticles have an average diameter between about 1μ and about 5μ.

15. The pharmaceutical composition of claim 9 wherein the microparticles are present in the pharmaceutical composition in a concentration of between about 20 mg/ml and about 300 mg/ml.

16. A pharmaceutical composition, suitable for intratumoral injection, comprising microparticles having an average diameter between about 2μ and about 4μ wherein the microparticles comprise from between about 65% by weight to about 75% by weight, based on the weight of microparticles, of paclitaxel and between about 25% by weight and about 35% by weight, based on the weight of microparticles, of polyvinylpyrrolidone.

17. A pharmaceutical composition, suitable for intratumoral injection, comprising microparticles wherein the microparticles comprise from about 50% by weight to about 75% by weight, based on the weight of microparticles, of paclitaxel, the remainder by weight of the microparticles comprising at least one water soluble polymer, wherein upon intratumoral injection of the composition paclitaxel is released intratumorally in a therapeutically effective amount in an extended manner for between about 24 and about 240 hours.

18. The pharmaceutical composition of claim 17 wherein the paclitaxel is released in a therapeutically effective amount in an extended manner for between about 48 and about 100 hours.

19. A method of treating a solid tumor comprising the step of intratumorally injecting microparticles, wherein the microparticles comprise from about 50% by weight to about 75% by weight of paclitaxel, the remainder by weight of the microparticles comprising at least one water soluble polymer.

20. The method of claim 19 wherein the water soluble polymer is selected from the group consisting of polyvinylpyrrolidone, hydroxypropylcellulose, methylcellulose, hydroxypropylmethylcellulose, sodium carboxymethylcellulose, hydroxyethylcellulose, and polysaccharides.

21. The method of claim 20 wherein the water soluble polymer is polyvinylpyrrolidone and the microparticles have an average diameter between about 2μ and about 4μ.

22. The method of claim 19 wherein, upon intratumoral injection of the microparticles, paclitaxel is released intratumorally in a therapeutically effective amount in an extended manner for between about 24 and about 240 hours.

23. The method of claim 22 wherein the paclitaxel is released in a therapeutically effective amount in an extended manner for between about 48 and about 100 hours.

24. The method of claim 19 wherein the microparticles are present in a pharmaceutical composition for intratumorally delivering the microparticles at a concentration of between about 100 mg/ml and about 300 mg/ml and the volume of pharmaceutical composition intratumorally injected is about 25% of the volume of the solid tumor.

25. The method of claim 23 wherein the solid tumor is selected from the group consisting of breast tumor, ovarian tumor, head and neck tumors, tumors of the peritoneal cavity, testicular tumors, tumors of the rectum, and pancreatic tumors.

26. The method of claim 25 wherein the solid tumor is a breast tumor.
